(19) United States
(12) Patent Application Publication
(10) Pub. No.: US 2007/0232949 A1
(43) Pub. Date: Oct. 4, 2007

(54) Method For Simultaneous Bi-Atrial Mapping Of Atrial Fibrillation

(75) Inventor: Sanjeev Saksena, Green Brook, NJ (US)

Correspondence Address: HANSEN HUANG TECHNOLOGY LAW GROUP, LLP, 1725 EYE STREET, NW, SUITE 300, WASHINGTON, DC, 20006, US

(73) Assignee: EP MedSystems, Inc., West Berlin, NJ (US)

(21) Appl. No.: 11/682,308

(22) Filed: Mar. 5, 2007

Related U.S. Application Data
(60) Provisional application No. 60/795,912, filed on Apr. 29, 2006.
(60) Provisional application No. 60/787,668, filed on Mar. 31, 2006.

(51) Int. Cl. A61B 5/0432

(52) U.S. Cl. 600/515; 607/27

(57) ABSTRACT

A method for diagnosing and mapping atrial fibrillation correlates recordings of electrical activity from intracardiac multielectrode catheters with the locations of electrodes within the heart to obtain a global mapping of cardiac electrical activity. Time delay and/or amplitude information in the recorded electrical activities is fused with electrode location information to generate a display on a 3-D anatomical template of the heart. Time delay and/or amplitude information is displayed using color code and/or lines of equal value, to aid diagnosis and localization of electrical activity irregularities. Mapping of atrial fibrillation enables physicians to treat arrhythmia by ablation, pacing, shock therapy and/or drugs at initiation or during an episode based on therapy delivery at critical mapped locations for arrhythmia onset or maintenance. Locations for placement of pacing leads and pacemaker timing parameters may also be obtained from the display.

### RELATED APPLICATIONS

This application claims priority to U.S. Provisional Application 60/795,912, filed Apr. 29, 2006, and U.S. Provisional Patent Application 60/787,668, filed Mar. 31, 2006. The entire contents of all of these previous applications are hereby incorporated by reference.

### FIELD OF THE INVENTION

This invention relates to medical diagnostic methods, and more particularly to methods for the detection and diagnosis of atrial fibrillation.

### BACKGROUND OF THE INVENTION

Atrial fibrillation is a disorganized electrical disorder of the upper chambers of the heart. It was once thought to be a disease of aging, relatively benign, and untreatable. However, the number of people exhibiting this disease is quite large, and the effects of the disease are quite profound. Atrial fibrillation presently affects over 2 million Americans, and this number is increasing with the aging of the population. It is the leading cause of stroke in the U.S., doubles the mortality from heart disease, and leads to reduced heart function. Thus, left untreated, atrial fibrillation leads to diminished lifestyle and serious morbidity and mortality. Thus, over the last several years, atrial fibrillation is a heart condition which has moved to the forefront in terms of both research, and clinically applied therapies. Research and developments in the area of recording and defining electrophysiological properties and anatomic locations of the tissue generating atrial arrhythmia have been published by the inventor, including U.S. Pat. No. 6,532,378 entitled “Pulmonary Artery Catheter for Left and Right Atrial Recording”, the entire contents of which are hereby incorporated by reference in their entirety.

Human physiological electrophysiological (EP) recording of the heart consists of “mapping” the timing of the activation of the various cells as very low voltage electrical activity conducts through the heart. To do this, various catheters with a plurality of recording electrodes are placed at various locations within the heart. In a basic study, catheters are placed in the high right atrium, the area around the atrioventricular (AV) node, and the apex of the right ventricle. These placements allow the physician to measure the conduction timing from the top of the heart to the bottom, primarily in the right atrium and right ventricle. To measure conduction from the right atrium to the left atrium, or laterally across the heart, a catheter, generally with a plurality of electrodes, is placed in the coronary sinus, a vessel which extends around the back side of the upper heart.

Recent research has shown that left atrial electrical activity is an important factor in the diagnosis of the origin of atrial fibrillation. Regional atrial mapping of different right and left atrial regions or very “focal” mapping of left sided electrical patterns from inside the atrium or pulmonary vessels is helpful. To enable such measurements, electrode catheters have been developed for placement in the left pulmonary artery for left atrial mapping, an example of which is described in U.S. Pat. No. 6,532,378, the entire contents of which are hereby incorporated by reference.

Current methods for mapping of the electrical activation in fibrillation (atrial or ventricular fibrillation) of the heart utilize sequential electrical signal acquisition and placement on previously constructed three-dimensional template or the use of the signals and catheter location for three-dimensional anatomic and electrical sequential mapping. Simultaneous mapping using a noncontact acquisition technique has been performed in the ventricles and in the atrium. However, this method allows only mapping of a single cardiac chamber at one time. However, fibrillation can arise in either chamber of the heart or occur independently in either chamber. Currently no method exists for simultaneous mapping of both upper chambers of the heart (bi-atrial mapping) with high-resolution three-dimensional mapping of the chamber of interest.

### SUMMARY OF THE INVENTION

The various embodiments provide methods for fusing electrical activity recordings obtained simultaneously at more than one location to obtain a global mapping of more than one location of a heart, such as bi-atrial or biventricular, with high-resolution three-dimensional mapping of electrical activity across most or the entire heart. This methodology allows physicians to obtain rapid information regarding the diseased electrical region of the heart and to perform detailed high-resolution mapping in that region of interest.

The various embodiments provide methods and systems which record electrical activity simultaneously from a plurality of electrodes positioned within or near both left and right atria or right and left ventricles and present electrical activity information on a display in a manner that reveals the time delay of electrical wave events across the 3-D surface of an anatomical template of the heart on a beat-to-beat basis.

The various embodiments may be used by a physician to identify a position on the heart for placement of a pacemaker pacing lead and for setting pacemaker parameters.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention.

FIG. 1 is a diagram of an electrophysiology catheter positioned within a heart.

FIG. 2 is a plan view of an electrophysiology catheter.

FIG. 3 is a system diagram of an electrophysiology system.

FIG. 4 is an example display of data according to an embodiment.

FIG. 5 is a detail of the example display of data shown in FIG. 4.

FIG. 6 is a detail of the example display of data shown in FIG. 4.

FIG. 7 is a detail of the example display of data shown in FIG. 4.

FIG. 8 is an example display of electrophysiological data that may be presented according to an embodiment.

FIG. 9 is a flow diagram of a method according to an embodiment.

FIG. 10 is a flow diagram of another method according to an embodiment.

### DETAILED DESCRIPTION OF THE PREFERRED EMBODIMENTS

The various embodiments will be described in detail with reference to the accompanying drawings. Wherever possible, the same reference numbers will be used throughout the drawings to refer to the same or like parts.

As used herein, the terms “about” or “approximately” for any numerical values or ranges indicates a suitable dimensional tolerance that allows the part or collection of components to function for its intended purpose as described herein. Also, as used herein, the terms “patient”, “host” and “subject” refer to any human or animal subject and are not intended to limit the systems or methods to human use, although use of the subject invention in a human patient represents a preferred embodiment.

The methods of the various embodiments enable physicians to obtain more complete and comprehensive visualizations of the activation of two separate chambers of the heart simultaneously so as to visualize real time electrical activation of heart chambers than possible with previously known methods. In the various embodiments, electrical activity recordings obtained simultaneously from two or more different locations of a heart are combined or “fused” to provide a global multi-section, such as bi-atrial or biventricular, measure of electrical activity which is mapped onto a high-resolution, three-dimensional (3-D) rendition of the chamber of interest. This methodology employing the fusion of electrical recordings and 3-D mapping information presents diagnostic information in a format that allows physicians to rapidly obtain information regarding diseased electrical regions of the heart while locating with high-resolution the regions of interest within the heart.

The proposed technique involves placement of multielectrode catheters simultaneously in two chambers of the heart (bi-atrial for the upper chambers, biventricular for the lower chambers) and measuring electrical activity in both chambers simultaneously. This bi-atrial mapping methodology uniquely allows for recording of electrograms on both chambers of the heart, with or without the use of electrode catheter placement using the concept of puncture methods. Electrode catheters suitable for placement in the left pulmonary artery for left atrial mapping are described in U.S. Pat. No. 6,532,378.

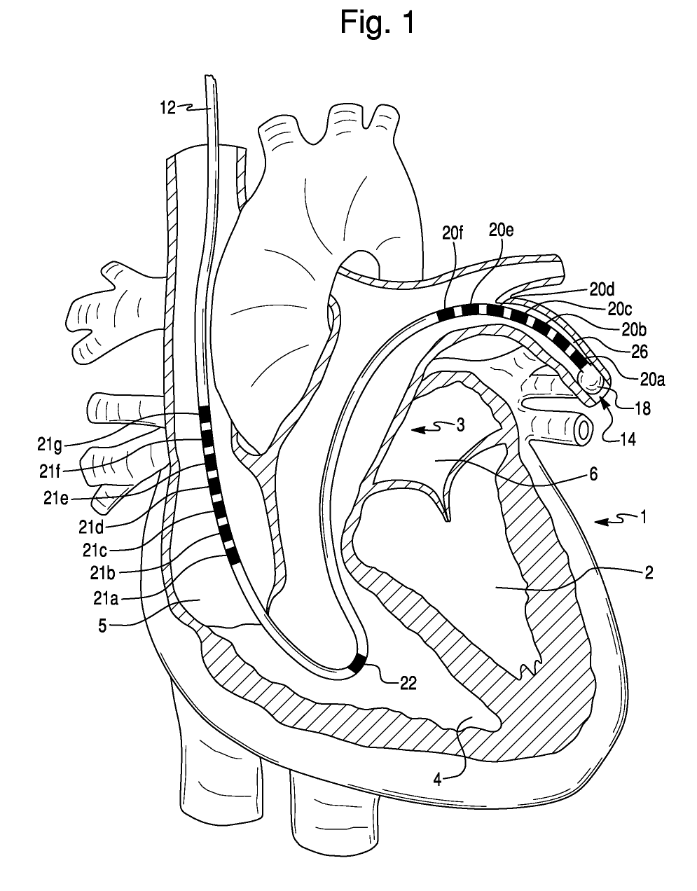

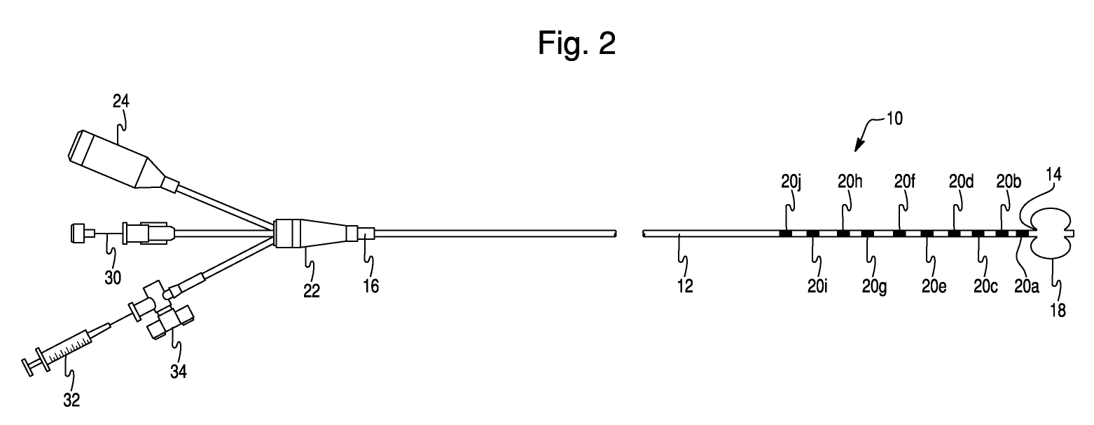

FIG. 1 shows a cross sectional view of a heart. The left atrium is partially blocked by the pulmonary artery in this view; nevertheless, the location of left atrium with respect to the pulmonary arteries is well known in medicine. Electrograms of electrical activity within the heart are obtained using electrode catheters positioned near or within the heart 1 as illustrated in FIG. 1. Such catheters include a flexible elongated member 12 with a distal end 14 and a proximal end 16, with an array of electrodes 20a-20j positioned on or near the distal end 14, (see also FIG. 2). A balloon 18 can be attached at the distal end 14 of the elongated member 12. The catheter 12 may also include additional electrodes 21a-21g located on the elongate member 12 a distance away from the distal end 14 so that when the distal end 14 is positioned in the vicinity of the left atrium 6, the additional electrodes 21a-21g are positioned in the vicinity of the right ventricle 4 and right atrium 5. The catheter may also include electrodes 22 located at other positions on the elongated member 12 in order to sense electrical activity at other locations in the heart, such as low in the right ventricle 2. The flexible member 12 may be made from extruded polyether block amide of the type sold by Atcochem North America, Inc. under the trademark PEBAX, but alternatively may be comprised of other polymeric materials with memory characteristics such as polyurethane, silicone rubber, and plasticized PVC etc.

Electrodes 20a-20j, 21a-21g may be spaced approximately 2 mm apart from each other on the catheter, with each electrode extending approximately 2 mm in length. Electrodes are preferably made of stainless steel, platinum, gold or other electrode material, and may be formed as thin flexible films applied to the exterior of the elongated member 12. The electrode array may extend over a length of approximately 35-40 mm of the elongated member 12. Electrical wires (not shown) from each electrode are positioned within and pass through the interior of the flexible member 12 to a manifold 22 secured to the proximal end 16 of the elongated member 12. Each electrode can be coupled to its own connector, which is shown, for example, at 24 in FIG. 2, and is ultimately connected to recording equipment located near the proximal end 16 of the elongated member 12.

As illustrated in FIG. 2, the catheter 10 may also include additional ports which may be used, for example, to introduce a guidewire 30 into the catheter, to attach an inflation mechanism for inflating the balloon, or to attach a syringe 32 with a stopcock 34 which may be used to introduce various solutions into the catheter.

Left atrial mapping can be performed by placing one (or more) multielectrode catheters in the left pulmonary artery and the coronary sinus. FIG. 1 illustrates one method for placing electrodes in a position to record left atrial electrical activity. In this method, the catheter is inserted into and guided through the heart as shown in FIG. 1 so that the distal end 14 of the catheter 12 with the mapping electrodes 20a-20f thereon are positioned within the left pulmonary artery 26. In this position, indirect left atrial mapping can be obtained in addition to mapping the superior interatrial septum, the superior left atrium 6, and the lateral left atrium 6. In addition, these recordings can detect early electrical activity in the right and left superior pulmonary veins. Coronary sinus electrodes can record inferior left atrium, mitral atrioventricular valve ring region and inferior pulmonary veins.

Right atrial mapping can be performed using multielectrode (with 12-48 electrodes) catheters in or near the right atrium. In an embodiment, electrodes 21a-21g located on the catheter 12 an appropriate distance from the distal end 14 can be positioned within the right atrium 5 when the catheter 12 is positioned in the heart 1 as illustrated in FIG. 1. Alternatively, another electrode catheter may be positioned in the right atrium 5, near the right interatrial septum or the superior right atrium, to record electrical activities. Recordings can also be obtained from the right pulmonary artery or other sections within or structures near the heart.

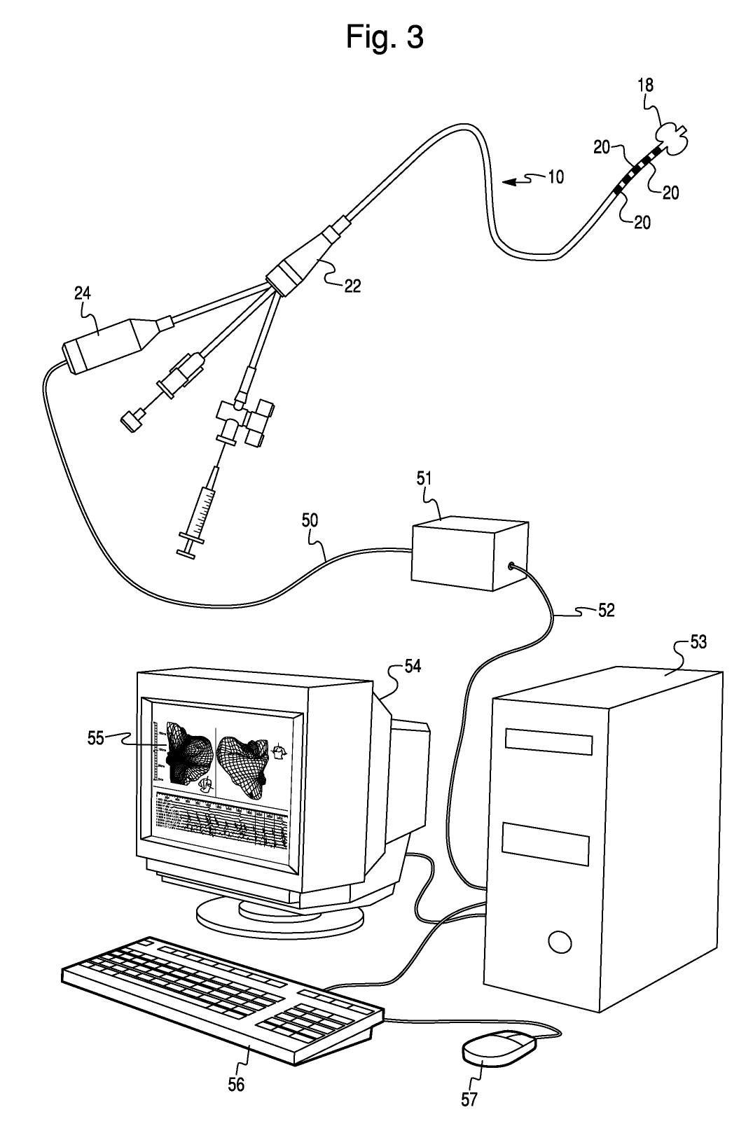

Electrical signals picked up by the plurality of electrodes are passed to an analyzer system as illustrated in FIG. 3. In this system, the catheter 10 is connected to an electrical isolation box 51, either directly (via connector 24) or via a cable 50 coupled between the connector 24 and the isolation box 51. The isolation box 51 includes circuits which isolate the patient from stray and fault currents which could be dangerous or fatal if conducted into the heart. Signals from the isolation box 51 are carried via a cable 52 to an analyzer 53 for amplification, processing and analysis. The analyzer 53 may include circuits for amplifying the electrical signals, removing stray (e.g., machine-induced and 60 Hz) noise, digitizing the signals and recording the information (e.g., on hard disc memory). The analyzer 53 also may include a processor (e.g., a microcomputer or microprocessor) programmed with software that allows it to perform the analysis methods of the present invention. Coupled to the analyzer 53 may be a monitor 54 which can present a graphical display 55 of the analyzed data as described more fully herein. Also coupled to the analyzer 53 may be user interface devices, such as a keyboard 56 and pointing device (e.g., a mouse) 57.

High-resolution 3-D mapping of measured electrical activity can be performed using acquisitions from existing high-resolution three-dimensional mapping techniques. An example of a suitable system that may be modified for performing the 3-D mapping is the WorkMate™ system manufactured by EP MedSystems, Inc. of West Berlin, N.J., although other systems may be used as well, such as any of the CARTO™, EnSite™, RPM™. Traditionally, such systems are employed for recording and mapping electrophysiology results from a single region of the heart. In various embodiments, electrical activity data is gathered simultaneously from numerous electrodes positioned across both atria of the heart, thereby measuring electrical activity of both atria simultaneously. Accordingly, the mapping systems may require modification in order to receive so much electrical activity data simultaneously and to display data for the entire heart.

The specific location of each electrode within or near the heart can be identified and correlated to an anatomical template (which may be a 3-D computer model or rendition) of the heart so that the electrical activity received by each electrode can be related to the corresponding location in the heart. The combination of multiple electrodes positioned in or near both atria with location mapping of each electrode to an anatomical template of the heart enables complete global mapping of the heart's electrical activity, which has not been possible with previously known systems.

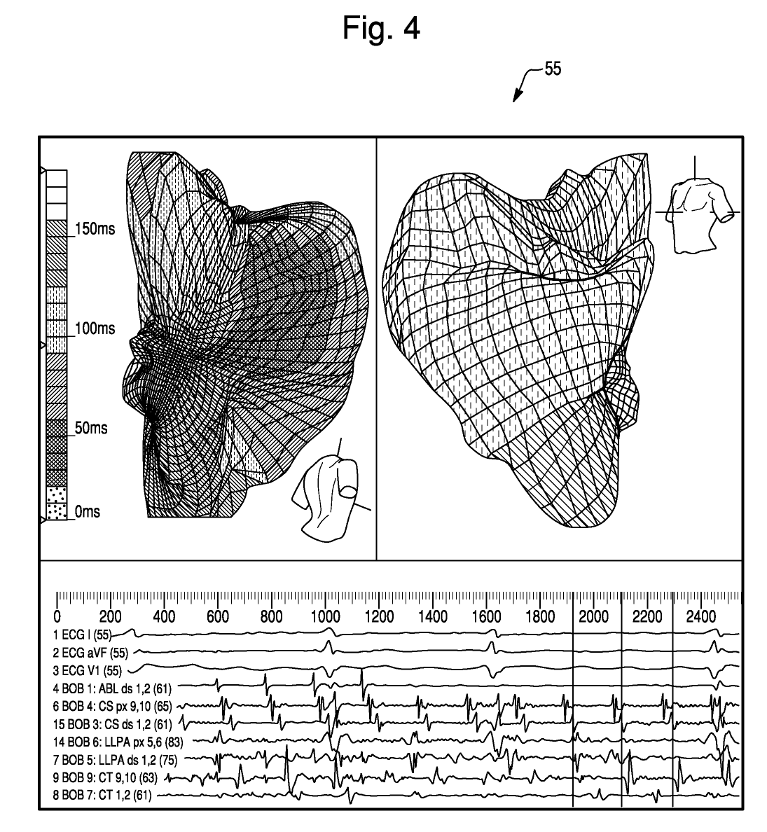

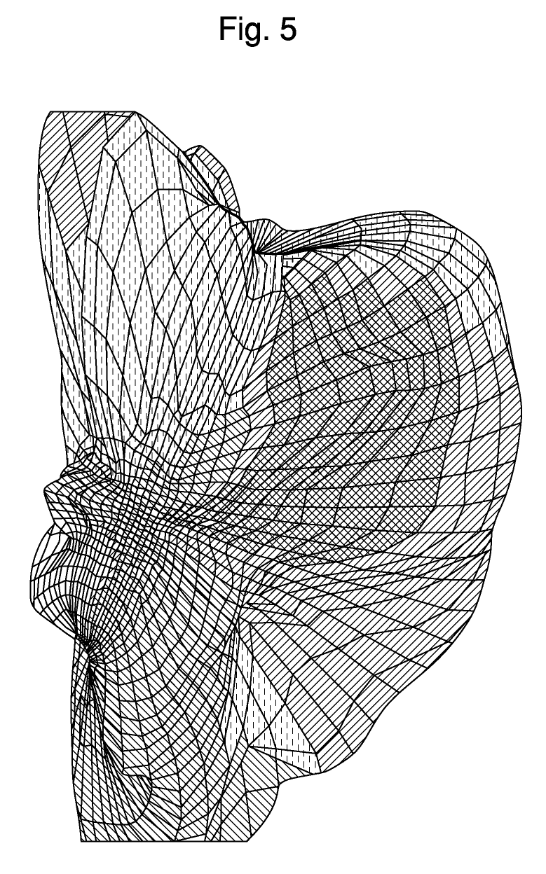

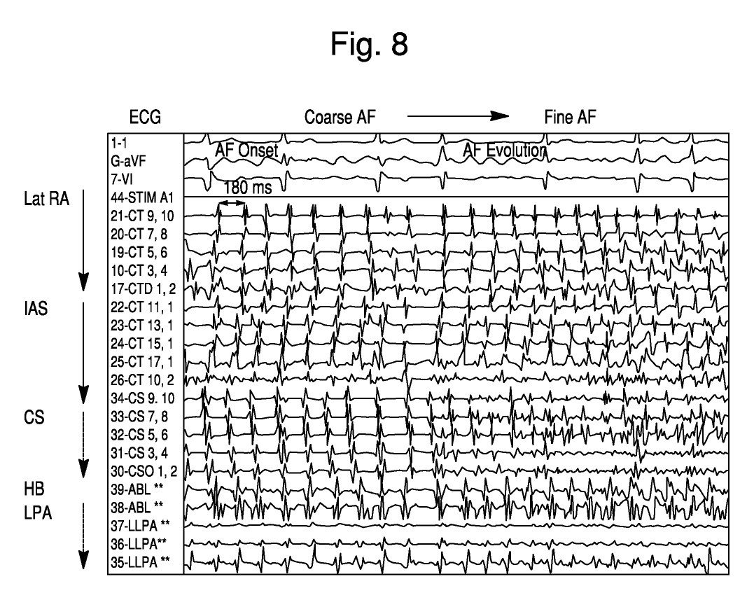

Once the individual electrode locations are mapped to an anatomical template of the heart, electrical activity at the various locations on the heart can be measured simultaneously, and the recorded electrical activity at each electrode correlated to the electrode locations (with respect to the heart) in both time and amplitude. Electrogram timing can be measured with reference to the surface electrocardiogram which can be obtained in the unipolar or bipolar modes. This information records the lag from electrode to electrode of each electrical wave passing through the heart, such as can be seen in the traces in FIG. 8. The result can then be used to generate a time-phased 3-D topographic map of the heart's electrical activity, an example of which is shown in FIGS. 4 and 5. The result is 3-D information on the electrical properties of the heart mapped over time, providing a four-dimensional (volumetric location plus time) data set, also referred to herein as a map. This data set can be stored in memory for use by the graphics processing software operating in the analyzer processor. This process involves recording electrical activity from each electrode for playback and analysis, and then displaying the activity (i.e., amplitude and timing) on a monitor so that the location, amplitude and timing information are conveyed to the physician. In order to convey all of the information being simultaneously recorded across the span of the heart, embodiments of the present invention employ graphical presentation techniques described herein, though other techniques may be used.

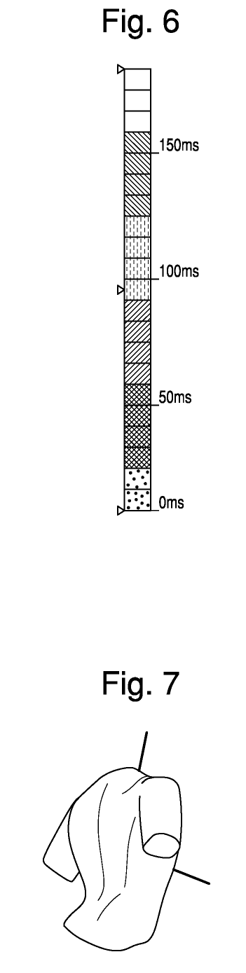

In an embodiment, the delay in the times that particular wave events are recorded at each electrode can be presented using color coding on the 3-D anatomical template. This can be understood by referring to the example time scale shown in FIG. 6 and the 3-D anatomic template shown in FIG. 5. Regions of the heart near an electrode that is first to sense a particular wave event can be color coded in white, pink or orange. Regions of the heart near electrodes that sense the same wave event delayed by between 50 and 100 milliseconds can be color coded in shades of yellow. Regions of the heart near electrodes that sense the same wave event delayed by between 100 and 150 milliseconds can be color coded in shades of blue. Regions of the heart near electrodes that sense the same wave event delayed by more than 150 milliseconds can be color coded by deep blue to purple. This manner of presenting the electrical activity provides a global map of the delay that enables visualization of the transmission of electrical activity across the heart.

In an embodiment which may be combined with the color coded embodiment above or employed alone, the system analyzes timing of particular electrical wave form events measured by each electrode as correlated to the 3-D anatomic template to identify surface lines of equal delay across the heart. These lines of equal delay, referred to as isochrones, are akin to lines of equal barometric pressure displayed on a weather map or lines of equal elevation displayed on a topographic map. For simplicity, each isochrone can represent an equal delay in the reception of a wave event (e.g., as a rising voltage) from the earliest recorded delay. Isochrones can be generated by the processor interpolating delay times between the locations of each sensing electrode and plotting the interpolated values on the 3-D anatomic template. Examples of isochrone lines are illustrated in FIGS. 4 and 5. Isochrones graphically reveal the electrical wave front as electrical impulses travel across the heart at each increment of time (i.e., the increment of delay). Isochrones can also identify nonessential electrical circuits in the heart enabling the physician to identify electrical irregularities and rotors (areas of activity maintenance) on the heart.

The combination of both the color coding and isochrone mapping on a 3-D anatomical template provides the physician with powerful analysis tools for assessing global cardiac electrical activity. This data may be presented in real time, allowing the physician to see the electrical waves moving across the heart on a beat-to-beat basis. The data may also be presented in slow motion or stopped in a “freeze frame” so that the transmission of electrical activity across the entire heart can be analyzed by the physician. Preferably, the data is presented over a number of beats of the heart so that the physician can observe how the global electrical patterns shift beat-to-beat. This information can show how arrhythmia initiates, changes and dies out beat-to-beat. This is important because there is increasing evidence that arrhythmia changes on each heart beat. The color coding and isochrone maps are therefore preferably updated on each beat. Viewed in slow motion, the physician can observe how each electrical wave starts, settles and stops. This information can be studied during a procedure, in regular speed, slow motion and/or stop motion to provide the physician with a complete picture of the heart's electrical activity before he continues with the rest of the procedure.

This biatrial mapping of electrical activity in real time on a 3-D anatomical template permits the physician to detect each location of irregular electrical activity. A common cause of problems with fibrillation treatments is the failure to identify and treat all centers of electrical dysfunction (e.g., rotors). Providing the physician with a display that will reveal all locations of irregular electrical activity can improve diagnoses and treatments since all centers of irregularity (e.g., rotors) can be detected before treatment is initiated. Displaying isochrones on a 3-D anatomical template will also reveal where nonessential electrical circuits are located in the patient's heart. Additionally, the mapping of electrical activity to the 3-D anatomical template permits the physician to accurately localize the areas of interest and concern. This capability can aid the physician in locating regions on the heart requiring pacing stimulus and thus in identifying locations for attaching pacemaker pacing leads. Additionally, by displaying time delay information, the capability can also aid the physician in setting pacemaker parameters (such as pace timing) for particular pacing leads. Such global mapping capabilities have not been possible with previously known systems.

In an example embodiment, one or more EP MedSystems, Inc. electrode catheters are placed in the right and left atria using standard cardiac catheterization techniques and connected to an EP MedSystems WorkMate™ system. The sites of the individual electrodes on each catheter are determined with respect to the heart and indicated on the anatomic template of the heart created by the Workmate™ system in the chamber of interest. Proper positioning of the electrodes within the heart can be confirmed by radiography, echo-location or by inspection of the signals received from electrodes as displayed on a monitor 54. With electrodes properly positioned and localized within the heart, electrogram recording can be initiated and the results displayed. Electrogram activation timing (i.e., the timing of particular electrical wave events) can be marked directly from the electrode catheters and the electrogram displayed on the 3-D anatomic template of the heart. Electrical signatures from regions in the right atrium and the left atrium may be specifically targeted. Timing information can be derived from features in the wave signals themselves, such as rising edges, falling edges, peaks, or valleys in the recorded electrical activity, examples of which are illustrated in FIG. 8.

Modifications to the display system required to implement various embodiments involve software additions to enhance the anatomical display template with software to import RPM images, and to superimpose the timing intervals and activation maps (e.g., color coding) onto the template.

Individual heart beat electrical cycles can be analyzed in the 3-D contour maps in which each of the bi-atrial map sites can be displayed, as well as the sequence of activation plotted on a 3-D anatomical template using isochrone and color coding indicia. A combined display for enabling such an analysis is illustrated in FIG. 4.

The three-dimensional mapping of electrical activity, such as illustrated in FIG. 4, permits high-resolution analysis of regions of interest in either chamber depending on the interest of the physician in the individual patient. In particular, this display allows the physician to visualize centers of origin or maintenance of electrical activity. Maintenance areas, which are areas where electrical activity persists or rotates within a confined area (referred to as “rotors”) can be a cause of fibrillation and can be treated by destroying part of the tissue in those areas using an ablation catheter. Prior known diagnostic systems did not provide sufficient information to allow a physician to identify all rotors simultaneously because they were limited to sensing and displaying specific areas sequentially and did not enable simultaneous visualization of global electrical patterns.

The example embodiment of a display shown in FIG. 4 combines and organizes the information obtained by the methods herein described in a fashion that is most useful to a physician. The display is organized with an anatomic template of the two atria and the two ventricles. Catheter electrode locations are marked on the template and the electrogram is displayed from the catheter electrode in real time.

In order to further display timing information, zones on the bi-atrial and biventricular templates can be colored, such as according to a displayed scale, an example of which is illustrated in FIG. 6. The result, shown in FIG. 5, is a 3-D display of the heart surface that reveals electrical wave front and phase lag information in a manner that allows the physician to identify, in a glance, areas of concern and their location on the heart. This display essentially provides a topographic map of electrical activity. While FIG. 5 is presented in black and white, a preferred embodiment displays timing information in color coding as described herein.

Beat-to-beat mapping can be performed so that an electrical activity topographic map is generated for each increment of a beat cycle, including from the beginning to the end of a fibrillation. Such color-coded maps may be viewed in real time, or slowed down to permit closer analysis of the electrical activity wave forms with each beat and from beat-to-beat. Since the electrical activity is mapped by the processor to a 3-D mathematical model of the heart, the display may also be rotated about various axes so that the electrical activity maps can be viewed from different angles in order to better obtain a global perspective of the heart. By seeing the entire heart from various perspectives and viewing how electrical activity changes from beat-to-beat, the physician is able to locate all areas in the heart where activity is initiated, maintained and/or dies out. The display also allows the physician to localize and track the evolution of the fibrillation, observe changes in the location and characteristics of rotors during the fibrillation event, and detect the development of the new rotors during the course of the fibrillation and from beat-to-beat. In this way, the physician is able to identify and localize all diseased tissue (i.e., correlate diseased tissue with particular anatomical features and locations on the heart) before treatments (e.g., ablation) are initiated. This avoids the potential that some areas of maintenance are overlooked and thus left untreated.

In order to aid the physician's analysis of the electrophysiology data presented on the display, each bi-atrial and biventricular templates can be rotated in any direction by use of a pointing device, the commands which are understood by the processor to generate rotated views of the templates. In order to show the physician the viewing perspective of a particular image, the display may also include torso models with X, Y and Z axis indicators aligned with the displayed bi-atrial and biventricular templates, an example of which is shown in FIGS. 4 and 7.

In addition to the 3-D graphical display of the electrophysiology information, the display may also include a standard trace display as illustrated in FIG. 8. Such a display may include the output of multiple physician-selected electrodes on a chart display that can be synchronized to the 3-D color coded electrical contour display described above with respect to FIGS. 4 and 5. This display can be calibrated to a time scale, such as time in milliseconds so that the timing relationships of electrical wave events can be accurately read from the display. By showing all of the electrode outputs from across the heart, the physician can identify and watch the evolution of atrial fibrillation. For example, FIG. 8 shows a clear transition from the atrial tachyarrhythmia at onset to course atrial fibrillation with no isoelectric period and then to sustained atrial fibrillation. Regions of the heart associated with individual electrode locations providing each trace are listed on the left of the figure, where “Lat RA” stands for lateral right atrium, “IAS” stands for intratrial septum, “CS” stands for coronary sinus, “HB” stands for His bundle, and “LPA” stands for left pulmonary artery. The arrows in FIG. 8 show how the recording exhibits high to low activation in the right atrium and medial to lateral activation in the left atrium recordings. In this example illustration, onset of atrial fibrillation is shown on the left side of the chart with subsequent evolution into sustained atrial fibrillation on the right hand side of the chart. Thus, viewing a display such as shown in FIG. 8, the physician can observe how the waveform of the atrial fibrillation evolves, such as becoming finer, displaying more fibrillatory conduction in the septum and coronary sinus regions as illustrated in FIG. 8. These areas may or may not be essential to the persistence of fibrillation.

An intracardiac reference electrode can also be selected for activation map timing. Such a reference electrode provides information related to reference electrical activity. This information can be used by the analyzer to reveal timing and average electrical activity.

The electrogram amplitudes can also be measured. Electrogram amplitudes reveal magnitude of the electrical activity at particular locations within the heart as a function of time. Such amplitudes may be included in the display as contour lines or color codes in a manner similar to those described above with respect to timing.

Analysis of the electrograms using a Fast Fourier Transform (FFT) technique at individual electrogram sites can be performed and electrograms selected for FFT analysis for frequency measurement. Frequency-domain analysis may be performed using known FFT algorithms implemented by the processor or in internal or external circuitry, such as commercially available digital signal processor integrated circuits (DSP chips). Performing frequency-domain analysis of the electrical activity across the heart and presenting the information in a 3-D display can be very useful in the diagnosis and treatment of fibrillation disorders. The frequency of an electrical impulse at a particular location and time in the heart can reveal the rate of conduction of impulses through the tissue at that location. Thus, FFT analysis of the electrograms can reveal locations of reduced electrical conduction that may be associated with diseased tissue and which may contribute to distortions or delays in the electrical conduction pattern over the surface of the heart. Also, the FFT analysis can be performed for each of a number of increments (or selected increments or phases) of the heart beat cycle to reveal how the rate of conduction varies in particular tissues with the electrical wave form features (e.g., rising or falling edges of each pulse) of each heart beat. Such frequency analysis of electrical activity can be conducted for each location in real time (or beat-to-beat) and mapped in four dimensions (locations on the heart plus time) to provide a frequency-domain map of electrical signals over the duration of a beat and over the surface of the heart. This map can be presented in a display similar to those described above, using color coding of frequency values or iso-frequency lines to present a useful display for the physician. In this manner, a frequency-domain analysis of the electric signals measured across the heart may reveal details about the locations and mechanisms of fibrillation and electrical dysfunction. Information obtained from a frequency-domain analysis can be stored along with or separate from the measured electrical activity for off-line access and archival purposes.

Additional analyses of measured electrical activity may be performed in order to obtain other diagnostically useful information. For example, various types and locations of data may be combined to calculate a figure of merit that is useful for diagnostic and therapy planning purposes. Since the measured electrical activity data are stored in a database, such additional analyses may be conducted in real-time or off-line.

Areas displaying electrical maintenance and rotors can be identified in individual regions of both atria and/or both ventricles of the heart. This method allows for simultaneous detection of more than one rotor as signals are acquired from different sites in the heart. Since most patients with atrial fibrillation have more than one rotor, this method can enable direct treatments of all rotors in one procedure, such as by means of catheter ablation.

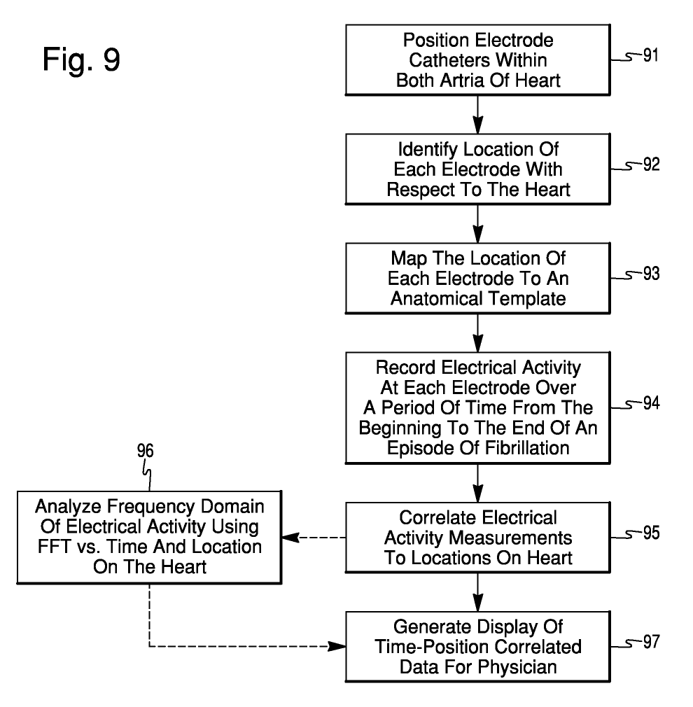

FIG. 9 presents a flow diagram of an embodiment of the present invention. In this embodiment, electrode catheters are positioned within the heart in both atria, step 91, and the locations of each electrode are determined with respect to the heart, step 92. With this information, the electrode positions are mapped to an anatomical template of the heart, step 93. Electrical activity of the heart is recorded from each electrode, step 94. Electrical activity recordings for each electrode are then correlated to the 3-D position of the respective electrodes against the anatomical template of the heart, step 95. Optionally, the electrical activity may also be analyzed using FFT algorithms (or signal processor chips) in step 96 to reveal frequency-domain information as a function of time and position on the heart. 3-D position, time, amplitude and (optionally) frequency information regarding the electrical activity is then used to generate a 3-D display for the physician in step 97, including displaying an activity, delay and frequency contour map of the heart.

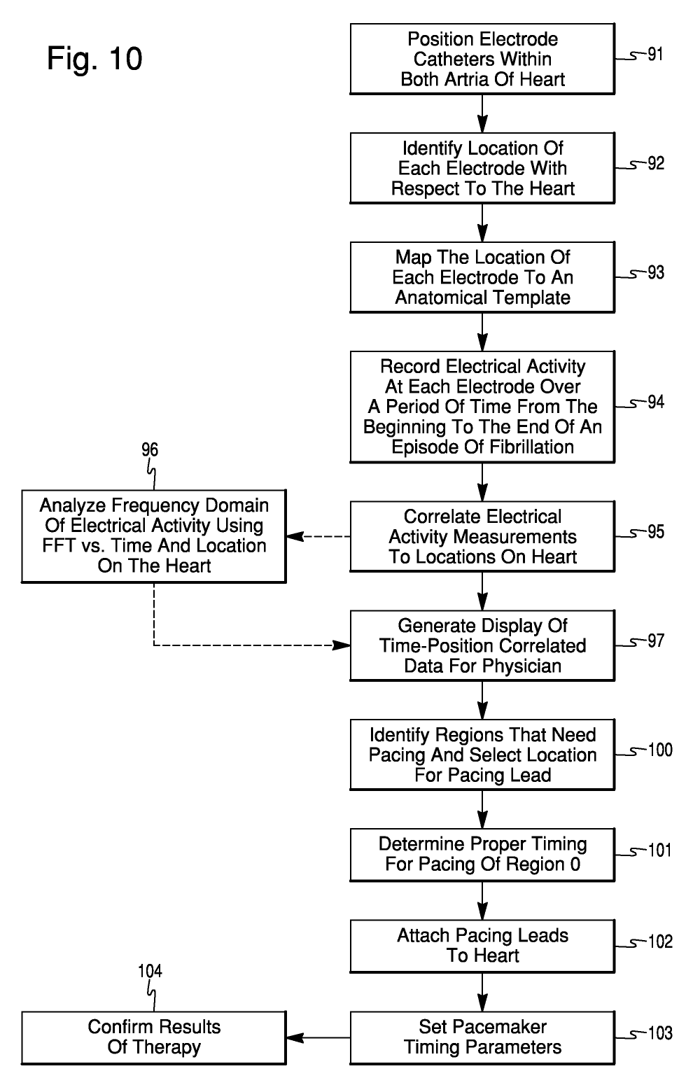

As discussed above, the various embodiments enable a physician to more locate a position on the heart for attaching a pacemaker pacing lead and for determining pacemaker parameters, such as a pacing time. FIG. 10 presents a flow diagram of an embodiment for a method of treating involving attaching pacing leads and programming a cardiac pacemaker. Referring to FIG. 10, electrode catheters are positioned within the heart in both atria, step 91, and the locations of each electrode are determined with respect to the heart, step 92. With this information, the electrode positions are mapped to an anatomical template of the heart, step 93. Electrical activity of the heart is recorded from each electrode, step 94. Electrical activity recordings for each electrode are then correlated to the 3-D position of the respective electrodes against the anatomical template of the heart, step 95. Optionally, the electrical activity may also be analyzed using FFT algorithms (or signal processor chips) in step 96 to reveal frequency-domain information as a function of time and position on the heart. 3-D position, time, amplitude and (optionally) frequency information regarding the electrical activity is then used to generate a 3-D display for the physician in step 97, including displaying an activity, delay and frequency contour map of the heart. Using the information presented in the 3-D display, the physician in step 100 identifies a region or regions of the heart that could benefit from pacing stimulus. Such regions may be revealed by lagging electrical activity (or reduced frequency from an FFT analysis) compared to adjoining tissue or an idealized model for electrical wave front propagation. Since the electrical activity is displayed on a 3-D map of the heart, the physician also use the display in step 100 to identify the specific location on the heart for attaching a pacing lead. Using the time or lag information presented on the 3-D map, the physician can also select an initial timing parameter for programming the pacemaker for a particular pacing lead in step 101. Armed with such information, the physician can proceed to attach a pacing lead or leads to the selected location on the heart, step 102, and set the pacemaker timing parameter with the determined setting, step 103. This can alter the electrical property so the atria so that atrial fibrillation does not recur. If the electrode catheters have been left in position while the pacing leads are attached and the pacemaker programs, then the steps of recording electrical activity to produce a display, steps 94-97, may be repeated to assess the electrical activity of the heart and confirm that the pacemaker therapy provides the desired therapeutic result or otherwise improves heart function, step 104. The foregoing procedure steps may be performed in different orders, and may be combined with other diagnostic methods, such as intracardiac ultrasonic imaging, to aid the physician in determining optimum pacing lead locations and pacemaker parameter programming.

Several aspects of the preferred embodiment methods are believed to have various advantages over previously known methodologies. The present method allows for beat-to-beat analysis of physiological electrical activity and related anatomical locations. The method enables rapid acquisition and detection of regions of electrical disease and rapid heart rhythm generation with high resolution three-dimensional localization performed online or immediately after acquisition. Regions in which rapid heart rhythm is initiated can become the target of immediate interventions such as ablation, pacing and drug therapy. This will allow for detection of the actual reach of these treatments on the regions of interest. The method enables detection and analysis of multiple regions of the heart involved in fibrillation simultaneous, as well as analysis of the electrical interactions of various regions of the heart during the same fibrillation event. The method enables beat-to-beat analysis of electrical activities from beginning through maintenance to termination of a fibrillation event with localizing correlation to the anatomical regions and features initiating, maintaining or terminating the event. This enables the physician to observe the beat-to-beat evolution of fibrillation, the change of rotors during fibrillation; how the fibrillation terminates.

The various embodiment methods allow for rapid acquisition and analysis during the procedure that it will reduce the time taken by a physician to perform an interventional electrophysiology procedure, thereby reducing trauma to the patient and costs of the procedure. Also, by reducing the duration of the procedure, this method reduces the amount of X-ray exposure to the patient and medical staff from fluoroscopy. By accurately localizing areas requiring therapy, such as ablation, the method enables effective treatments while minimizing the amount of heart tissue damaged by the treatment. Moreover, mapping of atrial fibrillation during onset, maintenance and termination enables the physician to facilitate termination of the arrhythmia by one or more therapies, including ablation, pacing, shock therapy and drugs, applied at initiation or during an episode based on therapy delivery at critical mapped locations for the arrhythmia onset or maintenance. The result is an overall refinement of treatments for fibrillation disorders.

Further disclosure of the present invention and discussion of clinical trials are provided in the article “Biatrial and Three-Dimensional Mapping of Spontaneous Atrial Arrhythmias in Patients with Refractory Atrial Fibrillation,” by S. Saksena, et al., Journal of Cardiovascular Electrophysiology, Vol. 16, No. 5, May 2005, the entire contents of which are hereby incorporated by reference.

While the present invention has been disclosed with reference to certain preferred embodiments, numerous modifications, alterations, and changes to the described embodiments are possible without departing from the sphere and scope of the present invention, as defined in the appended claims. Accordingly, it is intended that the present invention not be limited to the described embodiments, but that it have the full scope defined by the language of the following claims, and equivalents thereof.

What is claimed is:

1. A method for assaying a heart, comprising:
positioning at least one electrode catheter including a plurality of electrodes near or within the heart so that the plurality of electrodes are positioned to measure electrical activity at more than one location on the heart;
determining a location with respect to the heart of each of the plurality of electrodes;
mapping the location of each of the plurality of electrodes to an anatomical template of the heart;
measuring electrical activity at each of the plurality of electrodes simultaneously;
correlating measured electrical activity at each of the plurality of electrodes to the location of each electrode; and
generating a display of the measured electrical activity with respect to the anatomical template of the heart.

2. The method according to claim 1, wherein the at least one electrode catheter is positioned so that at least a subset of the plurality of electrodes are positioned across the right atrium and the left atrium to measure electrical activity in both atria simultaneously.

3. The method according to claim 2, wherein a time-delay of electrical activity at a particular location in the heart is presented in the display using color to show the time-delay on the anatomical template of the heart.

4. The method according to claim 3, wherein the display further includes isochrones to show the time-delay of electrical activity at particular locations on the anatomical template of the heart.

5. The method according to claim 2, wherein the anatomical template of the heart is a three-dimensional model of the heart.

6. The method according to claim 2, wherein measuring electrical activity is performed from the beginning of fibrillation until termination of fibrillation.

7. The method according to claim 2, further including analyzing measured electrical activity using a Fast Fourier Transform (FFT) algorithm and generating a display of measured electrical activity in the frequency domain.

8. The method according to claim 7, wherein the display of measured electrical activity in the frequency domain is mapped to the anatomical template of the heart.

9. The method according to claim 2, further comprising identifying a critical location on the heart for onset or maintenance of arrhythmia.

10. The method according to claim 9, wherein the locations are related to onset or maintenance of arrhythmia.

11. The method according to claim 10, further comprising delivering a therapy at the identified critical location on the heart.

12. The method according to claim 11, wherein the therapy is selected from the group consisting of ablation, pacing, shock therapy and local application of drugs.

13. The method according to claim 12, wherein the method is performed at initiation or during an episode of arrhythmia.

14. A method for treating arrhythmia, comprising:
positioning at least one electrode catheter including a plurality of electrodes near or within the heart so that the plurality of electrodes are positioned to measure electrical activity at more than one location on the heart;
determining a location with respect to the heart of each of the plurality of electrodes;
mapping the location of each of the plurality of electrodes to an anatomical template of the heart;
measuring electrical activity at each of the plurality of electrodes simultaneously;
correlating measured electrical activity at each of the plurality of electrodes to the location of each electrode;
generating a display of the measured electrical activity with respect to the anatomical template of the heart;
identifying on the display critical locations for onset or maintenance of arrhythmia; and
applying at the identified critical locations a therapy selected from the group consisting of ablation, pacing, shock therapy and local application of drugs.

15. The method according to claim 14, wherein the at least one electrode catheter is positioned so that at least a subset of the plurality of electrodes are positioned across the right atrium and the left atrium to measure electrical activity in both atria simultaneously.

16. The method for treating arrhythmia according to claim 15, wherein the method is performed at initiation or during an episode of arrhythmia.

17. A method of placing a pacemaker within a patient, comprising:
positioning at least one electrode catheter including a plurality of electrodes near or within the heart so that the plurality of electrodes are positioned to measure electrical activity at more than one location on the heart;
determining a location with respect to the heart of each of the plurality of electrodes;
mapping the location of each of the plurality of electrodes to an anatomical template of the heart;
measuring electrical activity at each of the plurality of electrodes simultaneously;
correlating measured electrical activity at each of the plurality of electrodes to the location of each electrode;
generating a display of the measured electrical activity with respect to the anatomical template of the heart;
identifying on the display a location on the heart that may benefit from pacing stimulation; and
attaching a pacing lead to the location on the heart.

18. The method according to claim 17, wherein the at least one electrode catheter is positioned so that at least a subset of the plurality of electrodes are positioned across the right atrium and the left atrium to measure electrical activity in both atria simultaneously.

19. The method for placing a pacemaker within a patient according to claim 18, further comprising:
determining from the display a suitable pacemaker operational parameter for the location that may benefit from pacing; and
programming the pacemaker with the operational parameter.

20. The method for placing a pacemaker within a patient according to claim 18, further comprising:
remeasuring electrical activity at each of the plurality of electrodes simultaneously;
correlating the remeasured electrical activity at each of the plurality of electrodes to the location of each electrode;
generating a display of the remeasured electrical activity with respect to the anatomical template of the heart; and
determine if pacing stimulation from the pacemaker benefits heart function.
